(19) United States
(12) Patent Application Publication
(10) Pub. No.: US 2026/0199620 A1
(43) Pub. Date: Jul. 16, 2026

(54) APPARATUS AND METHOD FOR INTUBATING AN AIRWAY OF A PATIENT

(71) Applicant: Anamed, LLC, Charlotte, NC (US)

(72) Inventor: Brendon Kipina, Charlotte, NC (US)

(73) Assignee: Anamed, LLC, Charlotte, NC (US)

(21) Appl. No.: 19/018,405

(22) Filed: Jan. 13, 2025

(51) Int. Cl. A61M 16/04

(52) U.S. Cl. CPC A61M 16/0497; A61M 2205/586

(57) ABSTRACT

An apparatus and method for intubating an airway of a patient. The apparatus includes a housing and a motive mechanism with a user interface structure. An endotracheal tube has proximal and distal ends fluidly connected by a ventilation lumen, wherein a stylet is slidably disposed. A tube coupler is supported by the housing and is adapted for motion relative to the housing. The tube coupler is operatively coupled to the motive mechanism and is connected to the proximal tube end. A stylet coupler is supported by the housing, operatively coupled to the motive mechanism and connectible to the stylet. The motive mechanism moves the tube coupler in a first direction and simultaneously moves the stylet coupler in an opposing second direction relative to the housing upon actuation of the user interface structure. The tube and stylet include features to promote smooth advancement of the tube into the patient's airway.

### BACKGROUND

### Field

The present disclosure relates generally to an apparatus and method for intubating an airway of a patient and, more particularly, to an intubation apparatus including an endotracheal tube with geometric features designed to facilitate advancement of the tube into the patient's airway, a stylet configured for use within the endotracheal tube, and a housing and coupler assembly enabling simultaneous advancement of the tube and retraction of the stylet in a single ergonomic movement by a user of the apparatus.

### Discussion of the Related Art

Endotracheal intubation is a medical procedure in which an endotracheal tube is placed in the trachea of a patient to facilitate breathing or to permit the controlled introduction of certain gases through the tube by an anesthesiologist or other medical personnel.

Trauma is a leading cause of death in the U.S. in patients between the ages of one and forty. The most common causes of trauma-related deaths are inadequate ventilation, inadequate circulation, or more massive hemorrhage for which there is little recourse. As critical care medicine developed over the past century, acute resuscitation techniques were established. Proper ventilation is important and may be very determinative of the outcome of a critical care event; if the patient cannot breathe, other critical care treatments may be provided in vain. However, it may be very difficult for a care provider to quickly and correctly intubate a patient in a high-stress situation, possibly with limited assistance. During the Vietnam War, for example, asphyxiation from upper airway obstruction or injury was a common cause of death in the field or enroute to forward surgical facilities. Even in non-emergency situations, such as providing a patient with anesthesia, oxygen, or other gasses in a surgical setting, it is desirable that the intubation of the patient occur quickly and efficiently and without damage to the throat structures of the patient. Medical providers therefore must consider airway management, breathing, and circulatory problems in both critical care and routine procedures. Unfortunately, medical device development in this area has lagged behind the recognized need.

In the most widely used protocol for intubating a patient (in both critical and routine applications), an endotracheal tube is inserted into the patient's airway with the assistance of a stiffening stylet. This stylet is generally a malleable metal rod placed within the lumen (interior opening) of the endotracheal tube to reinforce the structure of the tube, which would otherwise bend under pressure, and thereby help advance the tube through the patient's mouth and throat. Routinely, a laryngoscope is used to visualize the patient's airway and allow the user to directly observe the passage of the endotracheal tube and enclosed stylet, particularly in the initial stages of the procedure.

The stylet is used to stiffen the tube for passage through the patient's mouth and into the throat. However, it may be undesirable for the stylet to remain inside the tube past the vocal cords due to the potential for injury to delicate airway structures. Therefore, as the user is advancing the tube in the vicinity of the vocal cords with one (e.g., dominant) hand, normally with the assistance of a laryngoscope in the other (non-dominant) hand, an assistant is needed to at least partially withdraw the stylet from the tube. Once the user determines that the tube has been placed as desired, the stylet is completely removed from the tube (if not previously done) and ventilation of the patient commences.

This intubation process is resource-intensive and may be prone to errors. Particularly in a critical-care situation, the assistant is prevented from performing other, possibly time-sensitive, tasks while helping the user with intubation. The presence of two operators (the user and the assistant) could adversely affect the steadiness of the tube and/or stylet during the procedure, which may already be precarious if the intubation is being performed in a moving vehicle. Miscommunication could occur between the user and assistant, which could result in the stylet being withdrawn at a different speed and/or time than desired. The assistant could accidentally drag the tube out of position through the friction of the withdrawing stylet. Because of the delicate nature of the intubation procedure, any of these or other complications could substantially increase the time needed to perform the procedure and/or the risk of injury to the patient.

Even if the user sets aside the laryngoscope and grasps the stylet with the non-dominant hand, thus obviating the need for an assistant, it can be very difficult for a single person to simultaneously withdraw the stylet and to either hold the tube in position or continue advancing the tube, while accomplishing these tasks in a smooth and controlled manner.

Another problem commonly encountered during intubation procedures is the distal end of the endotracheal tube getting caught in a laryngeal notch in the throat of the patient during advancement of the tube. Reverse curvature of the tube is a known technique for overcoming this problem by “reverse loading” the tube onto the stylet—i.e., where the tube has a curvature (downward) opposite that of the stylet (upward). When reverse loaded, the tube is forced into a shape matching that of the much stiffer stylet, as long as the stylet remains within the tube. However, when the tube is loaded on the stylet in the reverse curvature position, the tube wants to rotate back to its natural preformed shape, particularly when the stylet is retracted from the distal end of the tube. This undesired rotation of the tube during intubation makes it difficult for the user to control the position of the distal end of the tube, which is problematic for reverse loading using existing devices.

In view of the circumstances described above, there is a need for an improved intubation apparatus which can be operated by a single user without assistance, facilitates smooth advancement of the tube into the patient's airway while maintaining desired curvature and trajectory, and enables simultaneous advancement of the tube and retraction of the stylet in a single ergonomic motion by the user.

### SUMMARY

The following disclosure describes an apparatus for intubating an airway of a patient with an endotracheal tube. The apparatus includes a housing and a motive mechanism supported by the housing and providing a user interface structure. The endotracheal tube has oppositely disposed proximal and distal ends fluidly connected by a ventilation lumen, wherein a stylet is slidably disposed. A tube coupler is supported by the housing and is adapted for motion relative to the housing. The tube coupler is operatively coupled to the motive mechanism and is connected to a proximal end of the endotracheal tube. A stylet coupler is also supported by and adapted for motive relative to the housing. The stylet coupler is operatively coupled to the motive mechanism and is connectible to a proximal end of the stylet. The motive mechanism is operable to move the tube coupler in a first direction relative to the housing and simultaneously move the stylet coupler in an opposing second direction relative to the housing in response to actuation of the user interface structure. The tube and stylet include geometric features to promote smooth advancement of the tube into the patient's airway. A method of intubating an airway of a patient using the apparatus is also described.

Additional features of the presently disclosed apparatus and methods will become apparent from the following description and appended claims, taken in conjunction with the accompanying drawings.

### BRIEF DESCRIPTION OF THE DRAWINGS

FIG. 1A is an illustration of an intubation apparatus in an initial configuration during early stages of intubating an airway of a patient with an endotracheal tube, and FIG. 1B is an illustration of the intubation apparatus in a final deployed configuration with the endotracheal tube fully advanced into the airway, according to embodiments of the present disclosure;

FIG. 2A is an illustration of the intubation apparatus of FIG. 1 in the initial configuration, and FIG. 2B is an illustration of the intubation apparatus of FIG. 1 in a final deployed configuration, depicting design details of the apparatus, according to embodiments of the present disclosure;

FIG. 3A is an illustration of a conventional endotracheal tube getting caught in a laryngeal notch in the throat of a patient during an intubation procedure, and FIG. 3B is an illustration of the endotracheal tube of the intubation apparatus of FIG. 1 in a final deployed configuration, depicting how reverse curvature of the tube facilitates successful traversal of the laryngeal notch, according to an embodiment of the present disclosure;

FIGS. 4A-4D are a sequence of illustrations depicting the reverse loading of the endotracheal tube of FIGS. 1, 2 and 3B onto the stylet of the same figures, according to embodiments of the present disclosure;

FIG. 5A is an illustration of a conventional endotracheal tube of the type shown in FIG. 3A, and FIG. 5B is an illustration of the endotracheal tube as shown in FIG. 3B, depicting design details enabling reverse loading the tube on the apparatus of FIG. 1, according to an embodiment of the present disclosure;

FIG. 6A is an illustration of a conventional stylet of the type shown in FIG. 3A, and FIG. 6B is an illustration of the stylet as shown in FIG. 3B, depicting design details enabling reverse loading the tube of FIG. 5B on the apparatus of FIG. 1, according to an embodiment of the present disclosure;

FIG. 7 is an illustration of the tube coupler and the tube connector components of the intubation apparatus depicted in FIGS. 1 and 2, showing how design features facilitate attachment and detachment of the tube coupler and the tube connector while preventing unwanted rotation of the proximal end of the endotracheal tube, according to embodiments of the present disclosure;

FIG. 8 is a flowchart diagram of a method for reverse loading the endotracheal tube of FIG. 5B onto the stylet of FIG. 6B, and intubating a patient using the apparatus of FIGS. 1 and 2 with the reverse loaded endotracheal tube, according to embodiments of the present disclosure;

FIGS. 9A and 9B are illustrations of an intubation apparatus with a simplified motion mechanism, used for intubating an airway of a patient with an endotracheal tube, according to another embodiment of the present disclosure; and

FIGS. 10A and 10B are illustrations of an intubation apparatus with no moving parts, used for intubating an airway of a patient with an endotracheal tube, according to yet another embodiment of the present disclosure.

### DETAILED DESCRIPTION OF THE EMBODIMENTS

The following discussion of the embodiments of the disclosure directed to an apparatus and method for intubating an airway of a patient is merely exemplary in nature, and is in no way intended to limit the disclosed devices and techniques or their applications or uses.

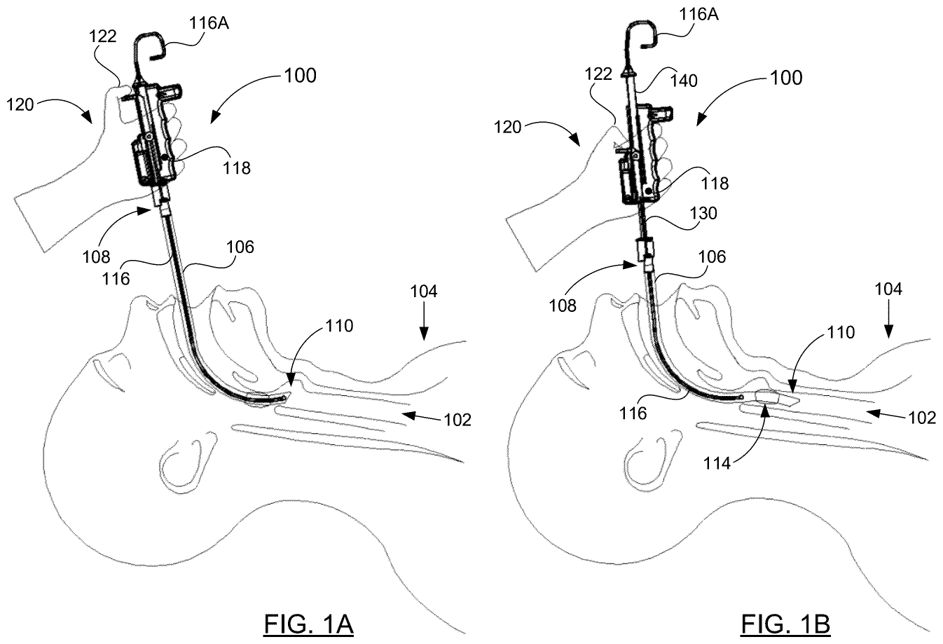

FIG. 1A is an illustration of an intubation apparatus 100 in an initial configuration during early stages of intubating an airway 102 of a patient 104 with an endotracheal tube 106, and FIG. 1B is an illustration of the intubation apparatus 100 in a final deployed configuration with the endotracheal tube 106 fully advanced into the airway 102, according to embodiments of the present disclosure.

The endotracheal tube 106 has oppositely disposed proximal and distal tube ends 108 and 110, respectively, fluidly connected by a ventilation lumen (interior opening). A cuff 114 (FIG. 1B) may be provided at or near the distal tube end 110 to assist in maintaining the endotracheal tube 106 in the desired position once intubation is complete and provide a seal between the endotracheal tube and the airway. The cuff 114 may be inflatable via a separate pressurization line provided with the endotracheal tube 106. The endotracheal tube 106 for use with the present invention may be a standard item or may include one or more specialized features for use with the apparatus 100. Like all structures described herein, the endotracheal tube 106 could be made of any material, and in any manner, as desired for a particular application of the present invention.

An intubation stylet 116 may be located at least partially within the ventilation lumen of the endotracheal tube 106, to help reinforce and shape the endotracheal tube 106 for insertion into the airway 102. The stylet 116 may be a standard item or may include one or more specialized features or structures for use with the apparatus 100. For example, the stylet 116 could be of the conventional malleable metal type, or could be a very rigid metal. Additionally or alternatively, the stylet 116 could include a light or a remote viewing device, such as a fiber optic camera, to facilitate visualization of the stylet or endotracheal tube 106 during intubation. The stylet 116 is also depicted and is more clearly visible in later figures. A stylet grasp loop 116A is formed into an end of the stylet 116 opposite the endotracheal tube 106. The stylet grasp loop 116A is useful for manipulating the stylet 116 relative to other parts of the apparatus 100.

The arrangement of FIG. 1A can be considered to show an initial or “primary intubation” configuration, in which the stylet/tube combination initially enters the airway 102 and is directed toward the vocal cords (omitted from FIG. 1A/B for clarity, but located generally in the area in which the distal tube end 110 is shown in FIG. 1A). In this initial configuration, the endotracheal tube 106 may be considered fully installed onto the stylet 116 for use, despite the extension of some portion of the stylet 116 from the proximal tube end 108.

FIGS. 1A and 1B provide an exterior view of the apparatus 100 which may be supplied integrally, in combination with, or separately from the endotracheal tube 106 and/or the stylet 116. For example, a reusable apparatus 100 could be packaged in combination with a plurality of single-use stylets 116 and/or endotracheal tubes 106. Whether sold separately or in a combination package, at least one of the stylet 116 and the endotracheal tube 106 may be specifically adapted for engagement with the apparatus 100. Examples of such specific designs and configurations are discussed below in connection with later figures. At least one of the stylet 116 and the endotracheal tube 106 may also or instead be a commercially available item. In addition, one of ordinary skill in the art could readily provide an adapter (not shown) to enable any suitable type of stylet 116 and/or endotracheal tube 106 to be used with the apparatus 100. The specific types of stylet 116 and endotracheal tube 106 which may be used with the apparatus 100 are not limited by the present disclosure.

As shown in the exterior view, the apparatus 100 includes a housing 118. The housing 118 may serve to enclose, support, and/or protect other structures of the apparatus 100. The material and/or configuration of the housing 118 may be chosen to provide desired properties, such as ease of sterilization. The housing 118 may be ergonomically shaped to at least partially mate with a hand 120 of a user and may include one or more cushioned and/or textured areas for ease and comfort in gripping, retention, and/or manipulation. The configuration of the housing 118 may be chosen in response to the shapes of one or more structures to be housed within the housing.

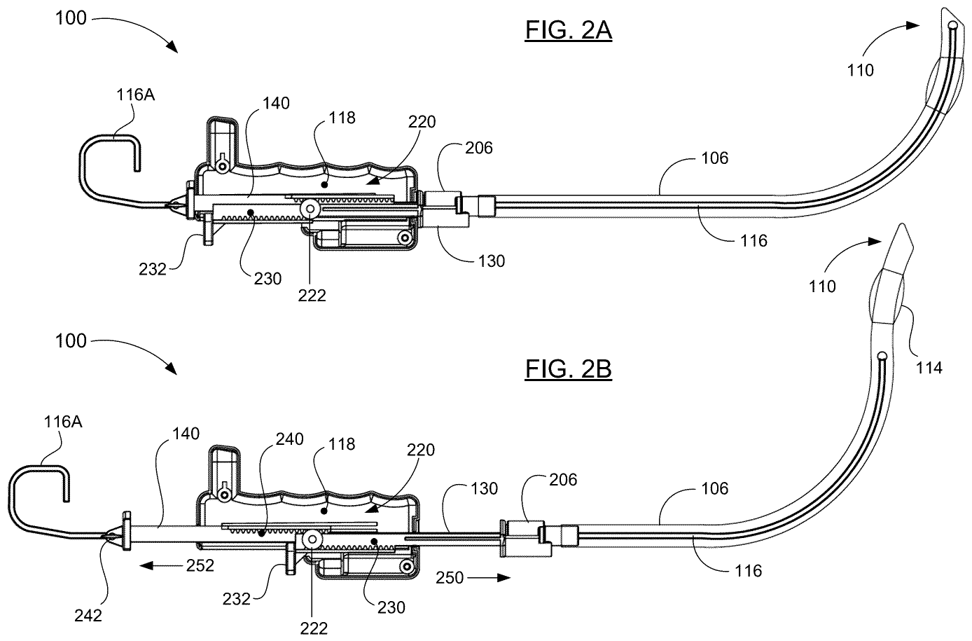

In a preferred embodiment, the hand 120 of the user grasps the housing 118 in the manner shown in FIGS. 1A and 1B, with fingers wrapped around a far side of the housing 118 and a thumb 122 placed on a slidable user interface structure (discussed below in connection with FIGS. 2A and 2B). This grasp position gives the user considerable leverage for maneuvering the apparatus 100 during the intubation procedure—particularly when compared to manipulating a conventional bare stylet. At an appropriate time during the intubation procedure (also discussed below), the user depresses the user interface structure with the thumb 122, causing motion of the endotracheal tube 106 in a first direction and simultaneous motion of the stylet 116 in a second direction opposite the first direction. This actuation motion advances the endotracheal tube 106 further into the airway 102 of the patient 104 while partially retracting the stylet 116 from the interior of the endotracheal tube 106. The extension of a portion of the endotracheal tube 106 beyond the end of the stylet 116 enables the endotracheal tube 106 to take on a downwardly-curved shape at the distal end 110, as shown in FIG. 1B. The downward curvature of the distal end 110 of the endotracheal tube 106 facilitates smooth advancement of the tube 106 into the airway 102 without getting caught on airway structures. These features of the apparatus 100 are also discussed in detail below.

The apparatus 100, or components thereof, may be provided in a range of sizes, shapes, and/or configurations for use with different stylets, endotracheal tubes, and/or user hand sizes/shapes. For example, left-handed and right-handed versions of the housing 118 may be provided, although this not necessary as the shape shown in figures fits comfortably in either a left or right hand.

A tube coupler 130 is supported by the housing 118 and is adapted for motion relative to the housing 118. The tube coupler 130 is connectable to the proximal tube end 108, in a manner discussed in detail below. A stylet coupler 140 is also supported by the housing 118 and is adapted for motion relative to the housing. The stylet coupler 140 is connectable to a proximal end of the stylet 116, and such connection may result from integral formation, or temporary or permanent attachment, of the stylet coupler 140 to at least a portion of the stylet 116.

In the deployed configuration of the apparatus 100 shown in FIG. 1B, portions of the tube coupler 130 and the stylet coupler 140 are visible outside the housing 118. The tube coupler 130 and the stylet coupler 140 are operatively coupled with the user interface structure to provide the deployment motion described above. In the initial configuration of FIG. 1A, the tube coupler 130 and the stylet coupler 140 are located predominantly within the housing 118. When the user interface structure is depressed by the thumb 122 as in FIG. 1B, the tube coupler 130 moves the endotracheal tube 106 in the first direction (further into the airway 102) while the stylet coupler 140 moves the stylet 116 in the second direction (retracting the stylet 116 from the endotracheal tube 106). The design and operation of these components, along with elements internal to the housing 118, is discussed further below.

FIG. 2A is an illustration of the intubation apparatus 100 in the initial configuration, and FIG. 2B is an illustration of the intubation apparatus 100 in a final deployed configuration, depicting design details of the apparatus 100, according to embodiments of the present disclosure. A near-side wall of the housing 118 is omitted from these views to facilitate depiction of the inner workings of the apparatus 100. FIG. 2A corresponds with FIG. 1A as discussed earlier - both being in the initial configuration where the stylet 116 is fully inserted in the endotracheal tube 106. Likewise, FIG. 2B corresponds with FIG. 1B—both being in the final deployed configuration where the stylet 116 is retracted from the distal end 110 the endotracheal tube 106.

Following is a list of elements of the intubation apparatus 100 and related components, as depicted in FIG. 1A/1B and 2A/2B.
100 intubation apparatus
102 airway
104 patient
106 endotracheal tube
108 proximal end of endotracheal tube
110 distal end of endotracheal tube
114 cuff
116 stylet
118 housing
120 hand
122 thumb
130 tube coupler
140 stylet coupler
206 tube connector
220 motive mechanism
222 pinion gear
230 tube rack
232 user interface structure
240 stylet rack
242 thumb screw
250 first direction of motion
252 second direction of motion

A tube connector 206 is provided at the proximal end 108 of the endotracheal tube 106. In preferred embodiments, the endotracheal tube 106 is provided as an assembly with the tube connector 206 attached. A preferred design embodiment is shown in a later figure. The tube connector 206 attaches to the tube coupler 130, such as by a snap fit, thereby enabling the apparatus 100 to apply force and motion to the endotracheal tube 106. In FIG. 2A, most of the tube coupler 130 is inside the housing 118, while in FIG. 2B more of the tube coupler 130 is visible outside the housing 118.

The stylet coupler 140 is also positioned mostly within the housing 118 in FIG. 2A, while in FIG. 2B more of the stylet coupler 140 is visible outside the housing 118. A thumb screw 242 is one embodiment of a means for operatively connecting the stylet 116 with the stylet coupler 140.

A motive mechanism 220 provides motion of the tube coupler 130 in a first direction 250 and simultaneous motion of the stylet coupler 140 in a second direction 252 upon activation of a user interface structure 232. The user interface structure and the first and second directions of motion were described in connection with FIGS. 1A and 1B.

The motive mechanism 220 is supported by the housing 118 and may be of any suitable type. The user interface structure 232 may be of any suitable type, such as the depicted “thumb actuated plunger” structure. The motive mechanism 220 moves the tube coupler 130 and the stylet coupler 140 in response to actuation of the user interface structure 232. The user interface structure 232 may be any structure which a user can manipulate, manually and/or automatically, to actuate the apparatus 100. For example, the user interface structure 232 could be a foot pedal, push button, remote control, squeeze bulb, slider, wheel, lever, touch sensor, electric or electromechanical switch, or the like, or any combination thereof. In various embodiments, the motive mechanism 220 and the user interface structure 232 may be actuated mechanically, electrically, electromechanically, or in any other suitable fashion.

In a preferred embodiment depicted in FIG. 2A/2B, the motive mechanism 220 comprises a pinion gear 222, a tube rack 230 and a stylet rack 240, where gear teeth of the pinion gear 222 engage with teeth of the tube rack 230 and the stylet rack 240. The pinion gear 222 is pivotably mounted to the housing 118. The tube rack 230 is integral with the tube coupler 130 (such as being formed as a single molded component, or fabricated separately and then attached), and the stylet rack 240 is likewise integral with the stylet coupler 140. The user interface structure 232 is also integral with the tube coupler 130. Thus, when the thumb 122 of the user depresses the user interface structure 232 (FIG. 1B), the tube coupler 130 extends outside the housing 118 in the first direction 250 as shown in FIG. 2B, the tube rack 230 causes rotation of the pinion gear 222, which in turn causes translation of stylet rack 240 and motion of the stylet coupler 140 in the second direction 252.

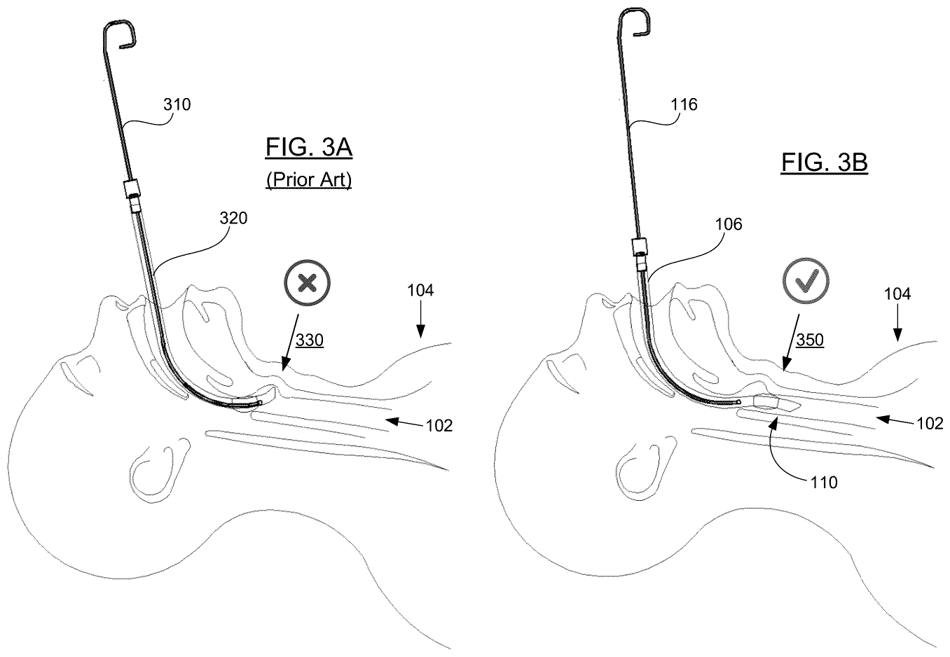

Regardless of how the tube coupler 130 and stylet coupler 140 are operatively coupled to the motive mechanism 220, the motive mechanism may be operable to move the tube coupler 130 in the first direction 250 relative to the housing 118 and to move the stylet coupler 140 in the second direction 252 relative to the housing 118, as shown in the actuated condition of FIG. 2B. (The housing 118 is considered to stay relatively stationary, as indicated by its constant position in FIGS. 3A and 3B.) In other words, the user interface structure 232 may transmit force and motion from a user to at least one of the tube coupler 130 and the stylet coupler 140 through the motive mechanism 220.

When the apparatus 100 is operatively connected to the endotracheal tube 106 and the stylet 116 and actuated via the user interface structure 232, the motions of the tube coupler 130 and the stylet coupler 140 act to advance the endotracheal tube 106 further into the airway 102 while withdrawing the stylet 116 from the distal tube end 110. The tube coupler 130 and the endotracheal tube 106 are moved in the first direction 250 substantially simultaneously with movement of the stylet coupler 140 and the stylet 116 in the second direction 252. The first direction 250 is substantially parallel to and opposite the second direction 252. The motive mechanism 220 may be used to move the tube coupler 130 and/or the stylet coupler 140 (and by extension the endotracheal tube 106 and/or the stylet 116) in any desired amount or manner suitable to provide the desired retraction of the stylet 116 from the endotracheal tube 106 as the endotracheal tube 106 is either maintained in position or advanced into the airway 102 of the patient 104. The design of the apparatus 100 also enables the user to apply other forces and motions to the stylet 116 and the endotracheal tube 106—such as prying motions to adjust the angle of attack of the distal tube end 110, etc.—separately from or simultaneously with the actuation of the user interface structure 232.

The motive mechanism 220 may include a mechanical advantage device (not shown), which could act to enhance and/or reduce the effect of the force exerted by the user upon certain structures of the apparatus 100. For example, a gear train could be provided in the motive mechanism 220, rather than the single pinion gear 222, and thereby provide a differential relationship between the movements of the user interface structure 232, the tube coupler 130, and/or the stylet coupler 140. The mechanical advantage device, when present, could use mechanical power, electrical power, electromechanical power, hydraulic power, pneumatic power, or the like, or any combination thereof, to provide desired ratios of motion and/or force between two or more of the user interface structure 232, the endotracheal tube 106, and the stylet 116. A suitable manual or automatic mechanical advantage device could be readily designed by one of ordinary skill in the art for a particular application of the presently disclosed devices.

It is contemplated that motion of the tube coupler 130 in the first direction 250 could at least partially disconnect the endotracheal tube 106 from the apparatus 100. For example, an ejector (not shown) could extend from the housing 118, and motion of the tube coupler 130 past the ejector could disengage the endotracheal tube 106 from the tube coupler 130. Such an ejector could facilitate complete separation of the apparatus 100 and stylet 116 from the endotracheal tube 106 while preserving one-handed use of the apparatus as desired.

One or more intubation accessories (not shown) could be provided to the apparatus 100, either integrally or in a separate or separable manner. For example, a suction device, an illumination device, a mechanical manipulation device (e.g., a blade), a remote viewing device (e.g., a fiber optic lens), a fluid supply device, a measuring device, and/or a remote access device (e.g., an endoscopic tool) could be provided. Any such intubation accessory could act at one or both of the proximal and distal tube ends 108 and 110, possibly with the assistance of the endotracheal tube 106, and could readily be provided by one of ordinary skill in the art.

In order to use any embodiment of the apparatus 100 to intubate the airway 102 of the patient 104, a stylet 116 is inserted into the ventilation lumen (internal opening) of the endotracheal tube 106. In many applications of the present invention, the stylet 116 will be provided in a pre-installed condition (stylet 116 already loaded through the housing 118 and attached to the stylet coupler 140), and the endotracheal tube 106 is loaded onto the stylet 116 as shown in later figures. However, the structure of a specific embodiment of the apparatus 100 may make any of the described steps desirable to perform in different sequences in different applications of the present invention.

The endotracheal tube 106 is connected to the tube coupler 130 via the tube connector 206, and the endotracheal tube 106 (with stylet 116 inserted) can be inserted into the airway 102 of the patient 104. The stylet 116 will stiffen and help guide the endotracheal tube 106 within the airway 102. The apparatus 100 may be manipulated by a single user to intubate the patient 104, with the user able to use just a single hand to perform the intubation due to the structural and motion features provided in certain embodiments of the apparatus 100. Any suitable intubation accessories (e.g., laryngoscope/blade, not shown) may be provided together or separately for use concurrently with the apparatus 100 to facilitate intubation.

Once the endotracheal tube 106 containing the stylet 116 has reached a desired level of insertion into the airway 102 (FIG. 1A), the user selectively actuates the user interface structure 232. In response to the user interface structure actuation, the tube coupler 130 moves in the first direction 250 relative to the housing 118 and the stylet coupler 140 moves in the second direction 252 relative to the housing 118. As a result of these relative movements, the stylet 116 is partially withdrawn from the endotracheal tube 106 (FIGS. 1B and 2B).

As discussed earlier, one problem which may be encountered in endotracheal intubation is the distal end of the endotracheal tube getting caught in a laryngeal notch in the throat of the patient during intubation, preventing further advancement of the tube. The devices of the present disclosure have been designed to overcome this problem, in addition to the advantages of one-handed operation of the apparatus 100 to control the position of the endotracheal tube 106 relative to the stylet 116.

FIG. 3A is an illustration of a conventional endotracheal tube getting caught in a laryngeal notch in the throat of a patient during an intubation procedure, and FIG. 3B is an illustration of the endotracheal tube 106 of the intubation apparatus 100 in a final deployed configuration, depicting how reverse curvature of the tube 106 facilitates successful traversal of the laryngeal notch, according to an embodiment of the present disclosure.

Because of the inherent shape of the human anatomy, the endotracheal tube must curve after passing through the patient's mouth in order to enter the airway 102. To facilitate the tube's navigation of this curve, stylets have a curvature (“concave upward and to the right” as seen in FIGS. 1 and 3). Endotracheal tubes are formed with a preset curvature generally matching the curvature of the stylet. Unfortunately, when using a conventional endotracheal tube and stylet as shown in FIG. 3A, the curvature of the stylet and endotracheal tube cause the distal tube end to project up into the laryngeal notch (an anatomical feature of the patient 104), where the distal tube end can become caught and unable to advance further into the airway 102 as needed. This situation is depicted at arrow 330 in FIG. 3A.

The apparatus 100 and the accompanying endotracheal tube 106 and stylet 116 of the present disclosure have been designed to overcome the problem described above and illustrated in FIG. 3A. This is accomplished by “reverse loading” the endotracheal tube 106 onto the stylet 116. Reverse loading the endotracheal tube 106 onto the stylet 116 causes the distal tube end 110 to curve away from the laryngeal notch when the stylet 116 is partially withdrawn, as shown in FIG. 3B and explained below.

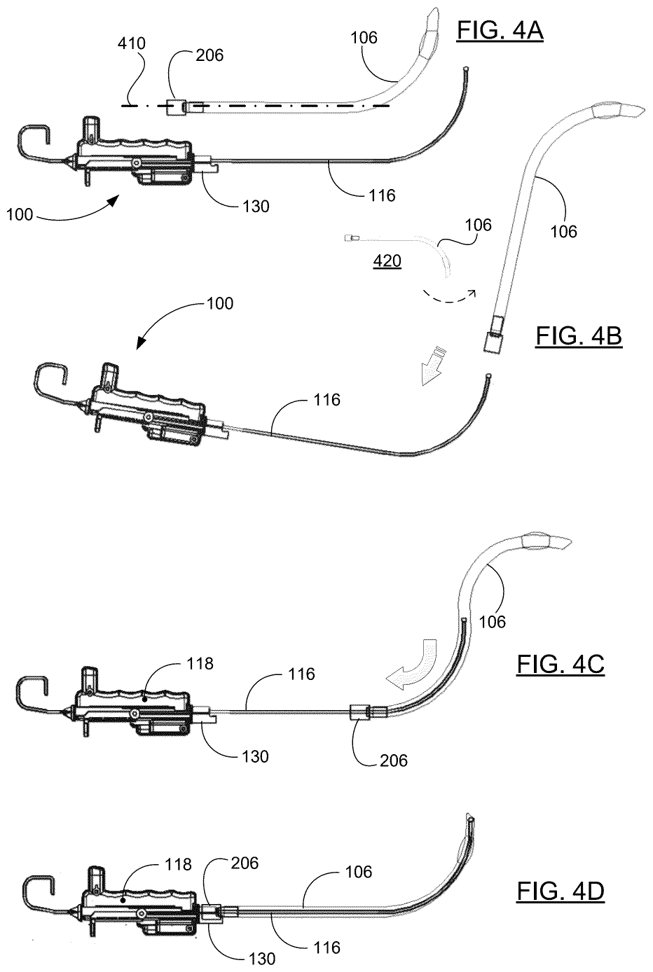

FIGS. 4A-4D are a sequence of illustrations depicting the reverse loading of the endotracheal tube 106 onto the stylet 116, according to embodiments of the present disclosure. In FIG. 4A, the endotracheal tube 106 and the stylet 116 of the present disclosure are shown in their initial configuration, “as taken out of the package”. The tube connector 206 is shown on the proximal end of the endotracheal tube 106, and the tube coupler 130 is shown proximal the housing 118 of the apparatus 100, in the manner discussed earlier.

In the initial configuration of FIG. 4A, the endotracheal tube 106 has a curvature generally matching that of the stylet 116. Reverse loading entails first rotating the endotracheal tube 106 about a central axis 410 to the position shown at 420, then sliding the endotracheal tube 106 onto the stylet 116 as shown in FIGS. 4B-4D. In FIG. 4B, both the apparatus 100 and the endotracheal tube 106 are rotated “in the plane of the page” to approximately align the endotracheal tube 106 with the tip of the stylet 116.

In FIG. 4C, the endotracheal tube 106 is shown partially loaded onto the stylet 116. Because the stylet 116 has much greater bending stiffness than the endotracheal tube 106, the endotracheal tube 106 takes on the curvature of the stylet 116. However, as can be seen at the top right of FIG. 4C, the endotracheal tube 106 is still in the reverse loaded orientation, with a predisposition to curvature opposite that of the stylet 116. Also seen in FIG. 4C is the tube coupler 130 proximal the housing 118.

In FIG. 4D, the endotracheal tube 106 is fully installed onto the stylet 116 and the tube connector 206 is fitted to the tube coupler 130. In this configuration shown in FIG. 4D, the apparatus 100 is ready for commencement of the intubation procedure, as shown in FIG. 1A.

As shown in FIG. 1B, when the stylet 116 is partially withdrawn from the endotracheal tube 106, the distal tube end 110 takes on a shape having a downwardly concave curvature—which is the natural pre-formed shape of the endotracheal tube 106, as can be understood from the sequence of illustrations in FIGS. 4A-4D. This same phenomenon can be seen in FIG. 2B as a curvature which is concave to the right.

Returning to FIG. 3B, where the stylet 116 is partially retracted from the endotracheal tube 106, it can be seen that the distal tube end 110 has taken on the downwardly-concave curvature as explained above, due to the reverse loading of the endotracheal tube 106. This downward curvature directs the distal tube end 110 away from the laryngeal notch and allows the endotracheal tube 106 to be advanced further into the airway 102 to the desired location, as indicated at arrow 350.

At that point (in the position shown in FIG. 3B), the endotracheal tube 106 can be disengaged from the apparatus 100 by uncoupling the tube connector 206 from the tube coupler 130, and the stylet 116 completely withdrawn, to place the endotracheal tube 106 into an available condition for ventilating the patient 104. Those portions of the apparatus 100, endotracheal tube 106, and/or stylet 116 which are reusable may be sterilized or otherwise prepared and/or stored for reuse, and the disposable portions may be discarded.

Reverse loading a conventional endotracheal tube onto a conventional stylet is possible in theory but not in practice because, during an intubation procedure, the endotracheal tube wants to rotate axially to take on its preformed shape with a curvature matching that of the stylet. This unwanted axial rotation of the endotracheal tube makes it very difficult for the operator performing the intubation to control the distal tube end.

The endotracheal tube 106, the stylet 116 and the apparatus 100 of the present disclosure have been designed with shape features which prevent unwanted endotracheal tube rotation after reverse loading. These features are shown in the following figures and discussed below.

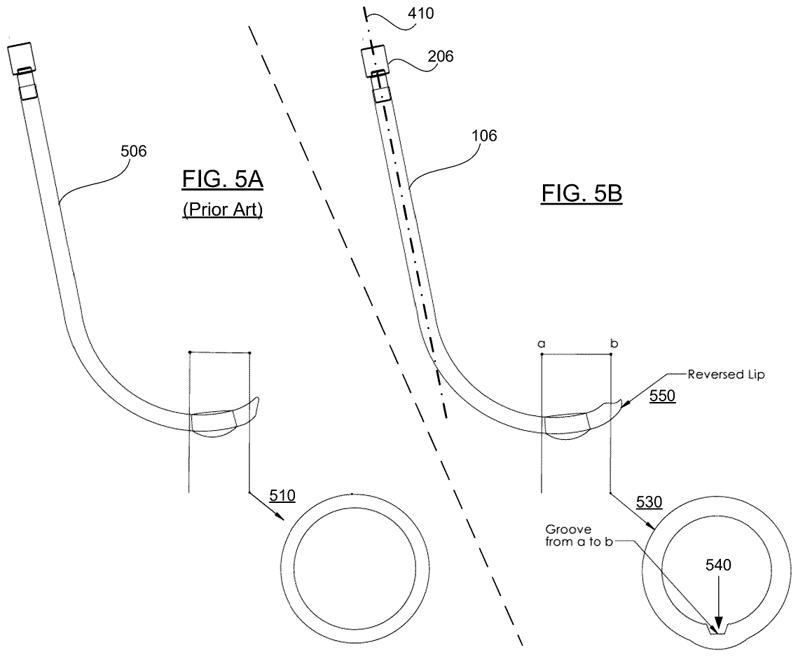

FIG. 5A is an illustration of a conventional endotracheal tube of the type shown in FIG. 3A, and FIG. 5B is an illustration of the endotracheal tube 106 as shown in FIG. 3B, depicting design details enabling reverse loading the tube 106 on the apparatus 100, according to an embodiment of the present disclosure.

A conventional endotracheal tube 506 is illustrated in a side view in FIG. 5A. The endotracheal tube 506 has a curvature matching a conventional stylet, as discussed previously with respect to FIG. 3A. A cross-section 510, taken near the distal tube end as shown, reveals that the conventional endotracheal tube 506 has an inner surface which is smooth and featureless. This is because there has heretofore been no reason for any geometric features on an endotracheal tube inner surface.

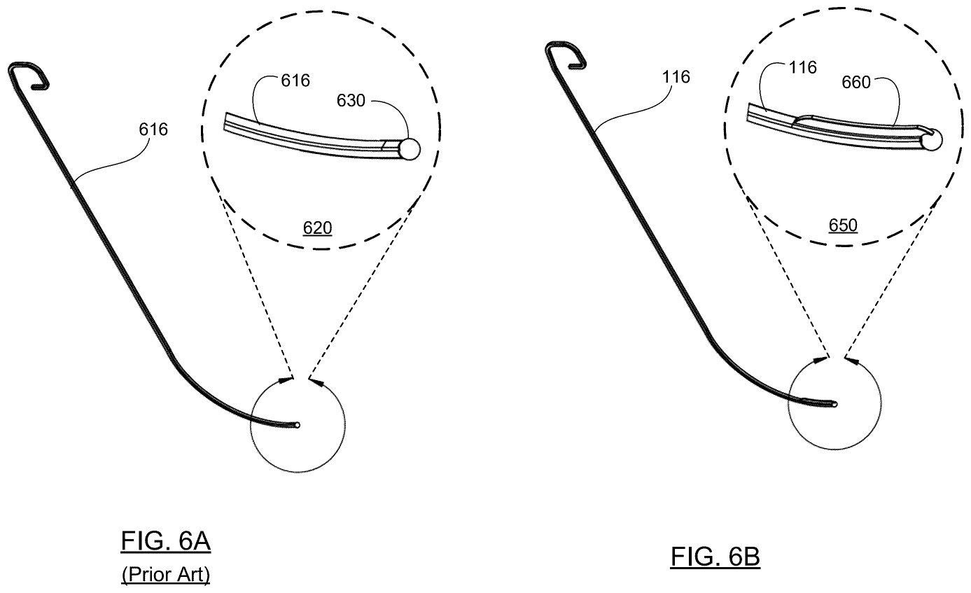

The endotracheal tube 106 of the present disclosure is illustrated in a side view in FIG. 5B. The endotracheal tube 106 is shown in its neutral (“just out of the package”) position, where it has a curvature matching the stylet 116, as discussed previously with respect to FIG. 4A. The central axis 410 of FIG. 4A is also shown again in FIG. 5B. A cross-section 530, taken near the distal tube end as shown, reveals that the endotracheal tube 106 has a groove 540 on the inner surface. The groove 540 exists at least in the distal end region of the endotracheal tube 106—such as from location “a” to location “b” as shown in FIG. 5B. The groove 540 may be longer or shorter than shown in FIG. 5B, and/or may exist at a second location along the length of the endotracheal tube 106. The groove 540 appears on the bottom of the cross-section 530. However, it must be kept in mind that in the process of reverse loading the endotracheal tube 106 onto the stylet 116, the endotracheal tube 106 will first be rotated about the central axis 410, and then rotated “in plane” to align with the stylet 116. After these rotations, the endotracheal tube 106 will have an orientation where the groove 540 is on the top of the cross-section when partially loaded onto the stylet 116 (as in FIG. 4C), and the groove 540 will align with a corresponding feature of the stylet 116 (see FIG. 6B) when the endotracheal tube 106 is fully loaded onto the stylet 116 (as in FIG. 4D).

The groove 540 engages with the corresponding feature of the stylet 116 to prevent the distal end of the endotracheal tube 106 from rotating relative to the stylet 116. The tube connector 206 is also shown in FIG. 5B, at the proximal end of the endotracheal tube 106. The tube connector 206 engages with corresponding features of the tube coupler 130 to prevent the proximal end of the endotracheal tube 106 from rotating relative to the stylet 116. These features enable reverse loading of the endotracheal tube 106 without the unwanted and problematic tube rotation relative to the stylet discussed earlier.

Also shown in FIG. 5B, at the very distal end of the endotracheal tube 106, is a lip 550. It can be seen that the lip 550 has a shape which is essentially reversed from that of the conventional endotracheal tube 506; this reversed shape of the lip 550 accounts for the rotation of the endotracheal tube 106 which is carried out during the reverse loading process. It is noted that the shape of the lip of the endotracheal tube 106 in any particular embodiment may be different from that shown in FIG. 5B, while still being compatible with the groove 540 as shown.

FIG. 6A is an illustration of a conventional stylet of the type shown in FIG. 3A, and FIG. 6B is an illustration of the stylet 116 as shown in FIG. 3B, depicting design details enabling reverse loading the endotracheal tube 106 of FIG. 5B on the apparatus of FIG. 1, according to an embodiment of the present disclosure.

A conventional stylet 616 is illustrated in a side view in FIG. 6A. An enlargement 620, taken near the distal end of the stylet 616 as shown, reveals that the conventional stylet 616 has a generally consistent and featureless cross-sectional shape, which may be square, rectangular or circular for example. In addition, a tip 630 having a spherical shape or the like, facilitates smooth advancement of an endotracheal tube onto the stylet 616 and prevents damage to delicate airway structures.

The stylet 116 of the present disclosure is illustrated in a side view in FIG. 6B. An enlargement 650, taken near the distal end as shown, reveals that the stylet 116 has a ridge 660 on its upper surface. The ridge 660 exists at least in the distal end region of the stylet 116—so as to correspond with the location of the groove 540 in the endotracheal tube 106 as shown in FIG. 5B when the endotracheal tube 106 is fully loaded onto the stylet 116 (as in FIG. 4D). The ridge 660 may be longer or shorter than shown in FIG. 6B, and/or may exist at a second location along the length of the stylet 116, so as to be compatible with the groove design in the endotracheal tube 106. The groove 540 of the endotracheal tube 106 engages with the ridge 660 of the stylet 116 to prevent the distal end of the endotracheal tube 106 from rotating relative to the stylet 116.

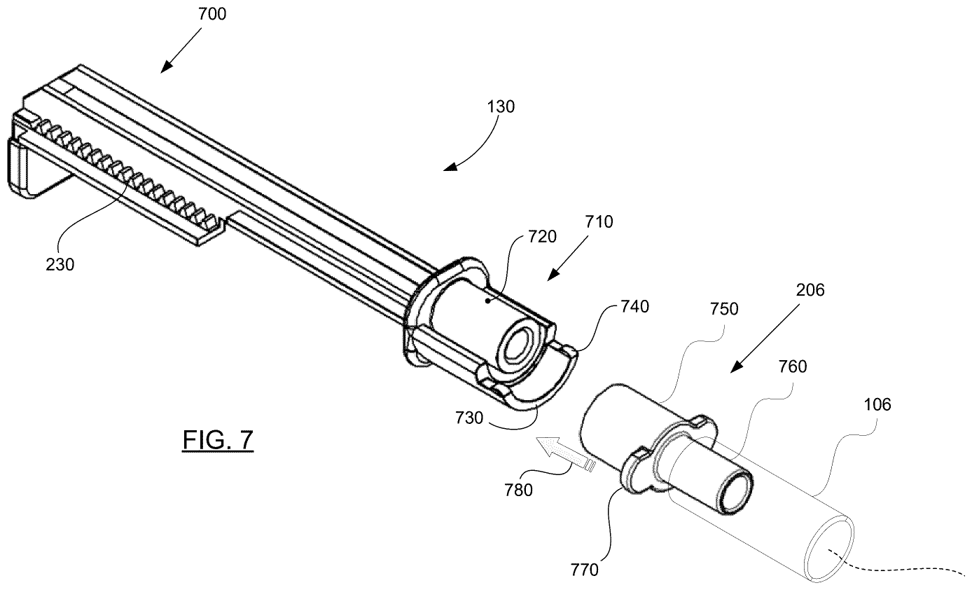

FIG. 7 is an illustration of the tube coupler 130 and the tube connector 206 as depicted in FIGS. 1 and 2, showing how design features facilitate tube coupler-connector attachment and detachment while also preventing unwanted rotation of the proximal end of the endotracheal tube 106, according to embodiments of the present disclosure.

The tube coupler 130 is shown on the left side of FIG. 7. The tube coupler 130 is the entire part having a long and slender shape, including the tube rack 230 (located inside the housing 118 as shown on FIG. 2) near a first end 700 and having a second end 710 with features for coupling to the tube connector 206 shown on the right side of FIG. 7. The tube coupler 130 may be formed as a single piece (such as by injection molding), or may be an assembly of two or more components.

At the second end 710, a cylindrical coupling 720 is sized to fit inside a mating component of the tube connector 206. The cylindrical coupling 720 has a central aperture through which the stylet 116 passes on its way to the endotracheal tube 106. Surrounding and spaced apart from the cylindrical coupling 720 is a partial cylindrical wall 730, at least the end portion of which spans an arc angle of less than 180° designed to fit with corresponding component features of the tube connector 206. On lateral edges of the partial cylindrical wall 730 are bumps 740 which are also designed for fit and engagement with the corresponding component features of the tube connector 206.

The tube connector 206 is preferably formed as a single part, such as by injection molding. The tube connector 206 includes a first cylindrical end 750 and a second cylindrical end 760. The first cylindrical end 750 fits over the cylindrical coupling 720 of the tube coupler 130, providing a connection with significant lateral force and lateral bending stability. The second cylindrical end 760 is sized to press-fit inside the endotracheal tube 106, which is typically made of a flexible plastic material such as from polyvinyl chloride (PVC), silicone, or polyurethane. A central aperture is formed through the tube connector 206 from the first cylindrical end 750 to the second cylindrical end 760, through which the stylet 116 passes on its way to the endotracheal tube 106.

The tube connector 206 also includes flanges 770, preferably on both lateral sides, at or near the junction of the first cylindrical end 750 and the second cylindrical end 760 as shown. The flanges 770 are designed to engage with the partial cylindrical wall 730 and the bumps 740 of the tube coupler 130. Specifically, when the tube connector 206 is moved and force is applied in the direction indicated by arrow 780, the flanges 770 snap-fit over the bumps 740; this completes the attachment of the tube connector 206 with the tube coupler 130. In this configuration, the flanges 770 abut the lateral edges of the partial cylindrical wall 730, which prevents axial rotation of the tube connector 206 relative to the tube coupler 130. In addition, the snap-fit of the flanges 770 over the bumps 740 provides a secure connection of the tube connector 206 with the tube coupler 130, preventing accidental detachment as the apparatus 100 is manipulated to complete the intubation procedure. After intubation is complete (the situation depicted in FIG. 1B), the tube connector 206 may be detached from the tube coupler 130 by the user purposefully applying a small separation force (conveniently applied to the flanges 770). At that point, the apparatus 100 is moved away and the stylet 116 is fully withdrawn from the endotracheal tube 106, and the tube 106 is used to ventilate the patient 104.

The elements 720, 730, 750 and even 760 need not be circular cylinders; they could be elliptical cylinders, or any other suitable geometric shape capable of the fit and function characteristics described above. Other designs for the tube coupler 130 and the tube connector 206 may be envisaged by those skilled in the art—as long as they enable the key features, including the physical extension of the tube coupler 130 from the housing 118, the passage of the stylet 116 through the tube coupler 130 and the tube connector 206, the mechanical attachment and detachment of the tube coupler 130 and the tube connector 206, the ability to withstand lateral forces and bending moments at the connection of the components, and the prevention of rotation of the tube connector 206 relative to the tube coupler 130.

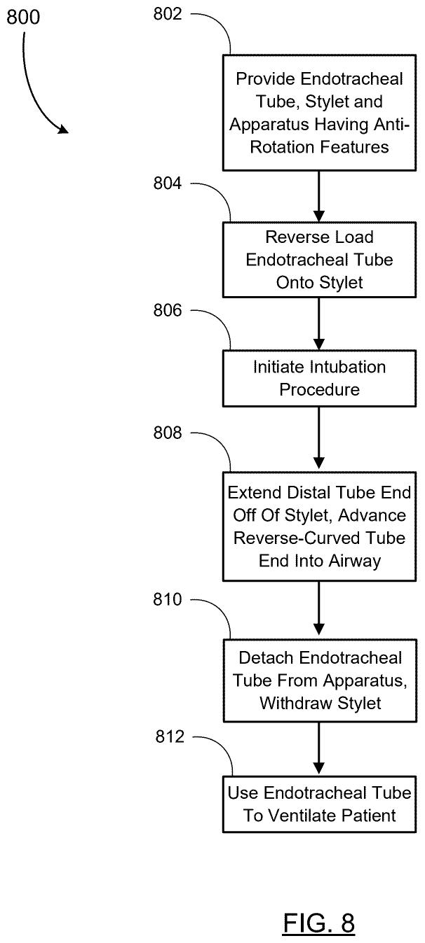

FIG. 8 is a flowchart diagram of a method for reverse loading the endotracheal tube 106 onto the stylet 116, as illustrated in FIGS. 4A-4D, and intubating a patient using the apparatus of FIGS. 1 and 2 with the reverse loaded endotracheal tube, according to embodiments of the present disclosure. At box 802, the endotracheal tube 106 and the stylet 116 are provided, having the anti-rotation features shown in FIGS. 5B, 6B and 7 as described above. Other endotracheal tube and stylet designs could be employed, as long as they include equivalent anti-rotation features—preferably preventing axial rotation of the endotracheal tube relative to the intubation apparatus at both the proximal end and the distal end of the endotracheal tube.

At box 804, the endotracheal tube 106 is reverse loaded onto the stylet 116 and coupled with the apparatus 100. This process was shown in the sequence of illustrations of FIGS. 4A-4D. As discussed in detail above, the endotracheal tube 106 is first rotated about its axis so that it has a curvature opposite that of the stylet 116. The endotracheal tube 106 is then loaded onto the stylet 116, whereupon the tube 106 takes on the shape of the stylet 116. The reverse loading is completed by attaching the tube connector 206 to the tube coupler 130, which at the same time causes engagement of the ridge 660 with the groove 540. The ridge/groove engagement prevents tube-stylet rotation at the distal tube end 110, and the attachment of the tube connector 206 to the tube coupler 130 prevents tube-stylet rotation at the proximal tube end 108.

At box 806, the intubation procedure is initiated using the apparatus 100 with the endotracheal tube 106 reverse loaded on the stylet 116. Initiation of the intubation procedure involves advancing the distal tube end 110 through the patient's mouth and toward the thoracic airway, as depicted in FIG. 1A. At box 808, the distal tube end 110 is extended off the end of the stylet 116, and the distal tube end 110 (now having a reverse-curved shape) is advanced into the patient's airway. The extension of the endotracheal tube 106 is advantageously performed, single-handedly, using the apparatus 100. It is to be understood that the extension of the distal tube end 110 off of the end of the stylet 116, and the advancement of the distal tube end 110 into the airway, may be performed gradually and incrementally, as the user sees and feels the progress of distal tube end advancement. FIG. 1B depicts the situation at the completion of the box 808.

At box 810, the endotracheal tube 106 is detached from the apparatus 100—such as by decoupling the tube connector 206 from the tube coupler 130 as described earlier in connection with FIG. 7. With the endotracheal tube 106 detached from the apparatus 100, the stylet 116 may be fully withdrawn from the endotracheal tube 106, also at the box 810. At box 812, the endotracheal tube 106 is used to ventilate the patient in the conventional manner.

The apparatus 100 described above—particularly with reference to FIGS. 1 and 2—provides numerous features for improving the efficiency and effectiveness of endotracheal intubation compared to existing devices and methods. However, other embodiments of an intubation apparatus are also envisioned—including the key features of reverse tube loading, positive control of the distal tube end position during tube advancement into the airway, and simple detachment of the endotracheal tube from the apparatus when intubation is complete. Some of these embodiments are discussed below.

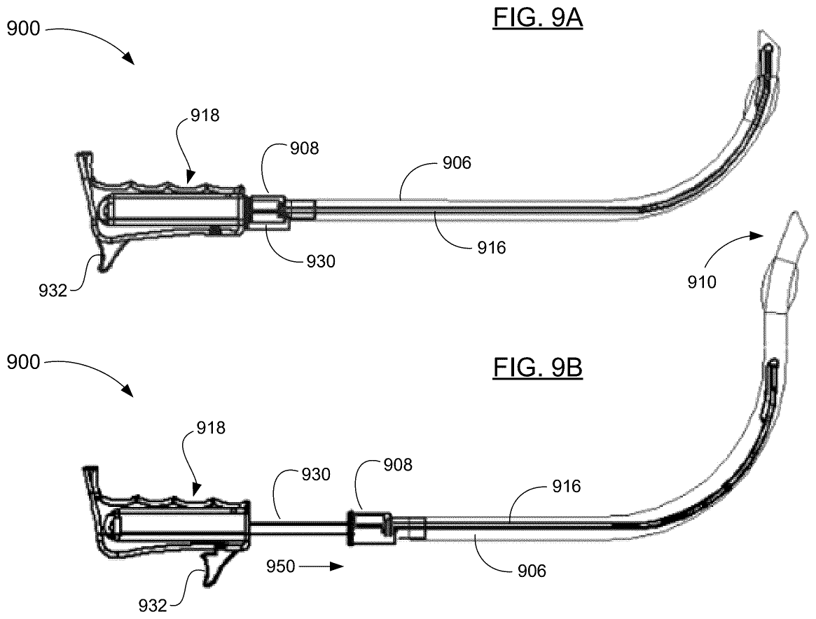

FIGS. 9A and 9B are illustrations of an intubation apparatus 900 with a simplified motion mechanism, used for intubating an airway of a patient with an endotracheal tube 906, according to another embodiment of the present disclosure. The intubation apparatus 900 has a simplified motion mechanism in comparison to the apparatus 100 depicted in FIGS. 1 and 2. Specifically, the intubation apparatus 900 includes only a single form of motion—a tube coupler 930 which may be extended from a housing 918, in a direction 950, by actuation of a user interface structure 932. The housing 918 of the apparatus 900 is designed to be grasped in the hand of a user, with the fingers of the user's hand (either hand) wrapped around one edge of the housing 918 and the thumb positioned along the opposite edge, where the thumb may be used to actuate the user interface structure 932 in the manner discussed earlier with respect to the apparatus 100.

The user interface structure 932 is located at a first end of the tube coupler 930 and is integral with the tube coupler 930; they may be formed as a single piece (e.g., injection molded), or formed separately and then attached. In any case, actuation of the user interface structure 932 from the initial position (FIG. 9A) to the deployed position (FIG. 9B) moves the tube coupler 930 in the direction 950. A second end of the tube coupler 930 attaches to a proximal tube end 908 of the endotracheal tube 906. Preferred embodiments for the attachment of the tube coupler 930 to the endotracheal tube 906 include the features and elements as depicted in FIG. 7—where a tube connector attaches to the tube coupler 930 with a light snap-fit, and the coupler-connector attachment prevents rotation of the endotracheal tube 906 with respect to the apparatus 900.

Unlike the apparatus 100, the apparatus 900 includes no mechanism for moving a stylet 916 with respect to the housing 918. Instead, the stylet 916 is fixed in the housing 918. This means that there is no stylet coupler, and no motive mechanism (e.g., racks and pinion gear) for coupling stylet motion to tube motion, in the apparatus 900. These differences make the apparatus 900 considerably simpler to manufacture and assemble than the apparatus 100.

The apparatus 900 provides the capabilities and benefits of reverse loading of the endotracheal tube as discussed earlier. That is, the tube coupler-connector attachment prevents rotation of the proximal end of the endotracheal tube 906 with respect to the apparatus 900, and the stylet 916 and the endotracheal tube 906 include the ridge and groove engagement shown in FIGS. 5 and 6. These features enable reverse tube loading by preventing unwanted rotation of the endotracheal tube 906 along its entire length.

As shown in FIG. 9B, when the tube coupler 930 is extended by actuation of the user interface structure 932, this moves the endotracheal tube 906 so that a distal tube end 910 deploys past the distal end of the stylet 916. This allows the endotracheal tube 906, when reverse loaded, to take on the reverse curvature shape which aids in advancement of the endotracheal tube 906 into the airway of the patient, as shown in FIG. 3A/3B and discussed earlier. The apparatus 900 can also be used with conventionally-loaded (not reverse loaded) endotracheal tubes.

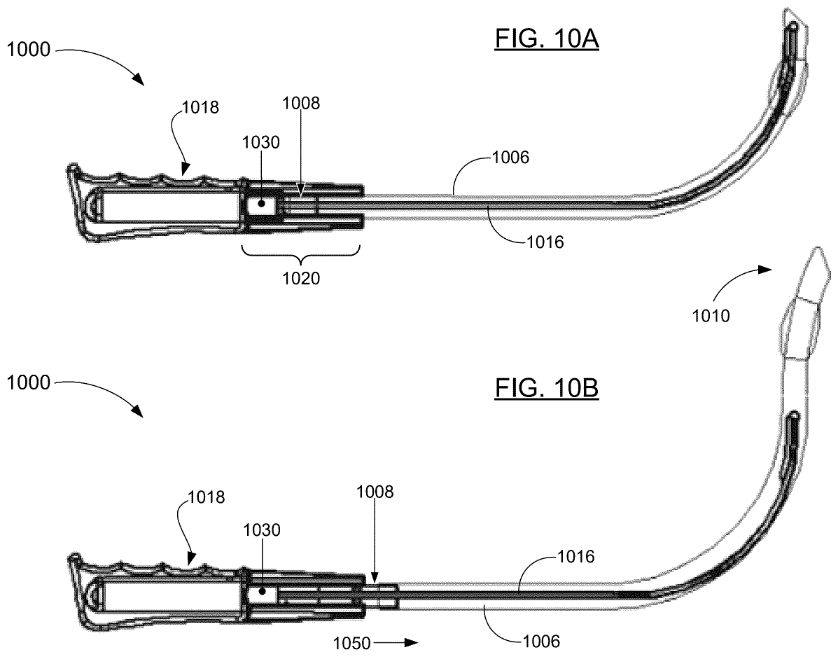

FIGS. 10A and 10B are illustrations of an intubation apparatus 1000 with no moving parts, used for intubating an airway of a patient with an endotracheal tube, according to yet another embodiment of the present disclosure. The intubation apparatus 1000 has a housing 1018 with an ergonomic design similar to the housings of the apparatus 100 and the apparatus 900—having a shape which conforms to a user's hand with the fingers wrapped around one edge. With the intubation apparatus 1000, there is no user interface structure, so the user's thumb can be placed in any location on the housing 1018 based on comfort and grip security.

The intubation apparatus 1000 has a tube coupler 1030 designed for attachment of a proximal end 1008 of an endotracheal tube 1006. The proximal end 1008 includes a tube connector in the manner discussed earlier. The tube coupler 1030 is integral with the housing 1018 and does not move relative to the housing 1018. Instead, in order to deploy the endotracheal tube 1006 off the end of a stylet 1016, the user simply grasps the proximal tube end 1008 (i.e., the tube connector) with his/her other hand, pushes the proximal tube end 1008 off of the tube coupler 1030 and moves the endotracheal tube 1006 in a direction 1050. At that point, a distal tube end 1010 begins to take the reverse curvature shape as explained earlier, and the endotracheal tube 1006 can be smoothly advanced further into the airway of the patient. Uncoupling the proximal tube end 1008 from the tube coupler 1030 could of course be performed by an assistant rather than the user's other hand.

Preferred embodiments for the attachment of the tube coupler 1030 to the endotracheal tube 1006 include the features and elements as depicted in FIG. 7—where a tube connector attaches to the tube coupler 1030 with a light snap-fit, and the coupler-connector attachment prevents rotation of the endotracheal tube 1006 with respect to the apparatus 1000. These features, along with the stylet 1016 having a ridge as depicted in FIG. 6B and the endotracheal tube 1006 having a groove as depicted in FIG. 5B, enable reverse loading the endotracheal tube 1006 onto the stylet 1016 and the apparatus 1000. The apparatus 1000 can also be used with conventionally-loaded endotracheal tubes.

In addition, the housing 1018 includes an extension section 1020 (FIG. 10A) which prevents rotation of the proximal tube end 1008 for some distance in the direction 1050 as the endotracheal tube 1006 moves away from the tube coupler 1030. The extension section 1020 helps the user maintain control of the orientation of the endotracheal tube 1006 as the tube 1006 is advanced into the airway of the patient. In one embodiment, the proximal tube end 1008 includes a tube connector having flanges 770 as shown in FIG. 7, and the extension section 1020 comprises a fork-like structure with upper and lower arcuate blades which surround the flanges 770 and prevent rotation of the proximal tube end 1008 as it is initially moved away from the tube coupler 1030.

The housing 1018 may be formed as a single component, such as by injection molding, or may be assembled from two or more components. In addition, other designs may be envisioned for the extension section 1020 and a corresponding tube connector. An intubation apparatus with no moving parts and having these features can be provided very cost-effectively—while still enabling reverse loading of the endotracheal tube onto the stylet and providing positive positional control throughout the intubation procedure.

While aspects of the presently disclosed devices and methods have been particularly shown and described above with reference to the preferred embodiments, it will be understood by those of ordinary skill in the art that various additional embodiments may be contemplated without departing from the spirit and scope of the present invention. For example, various components could be combined into a single element, and/or individual components couple be separated into multiple elements. Additional structures could be present in the apparatus 100 to assist in guiding, anchoring, steadying, connecting, or otherwise manipulating the described structures as desired. The described structures could include slots, bends, or any other features operative to prevent physical interference with other structures of the apparatus 100. Any suitable motive mechanism could be used, as long as the chosen structure is operative to move the tube coupler 130 and/or stylet coupler 140 as described. The initial conditions and actuated conditions depicted do not limit the possible arrangements in which the apparatus 100 may be placed; there are a plurality of intermediate arrangements between and/or beyond the initial and actuated conditions shown in which the apparatus 100 may be placed or used. The patient could be human or could be a non-human animal, with appropriate structural modifications to the apparatus 100 as needed. A device or method incorporating any of these features should be understood to fall under the scope of the present invention as determined based upon the claims below and any equivalents thereof.

The foregoing discussion discloses and describes merely exemplary embodiments of the intubation devices and methods. One skilled in the art will readily recognize from such discussion and from the accompanying drawings and claims that various changes, modifications and variations can be made therein without departing from the spirit and scope of the disclosure as defined in the following claims.

What is claimed is:

1. An apparatus for intubating an airway of a patient with an endotracheal tube, said apparatus comprising:
a housing; and
a tube coupler supported by the housing and adapted for slidable motion relative to the housing, the tube coupler being connectable to a proximal end of the endotracheal tube,
where the tube coupler is adapted to allow slidable passage of a stylet through an aperture of the tube coupler and into the proximal end of the endotracheal tube,
and where the tube coupler has a first end with a user interface structure operable to move the tube coupler in a first direction relative to the housing in response to actuation of the user interface structure,
and the tube coupler has a second end with a fitting for connection to the proximal end of the endotracheal tube, the fitting being adapted to prevent rotation of the proximal end of the endotracheal tube relative to the tube coupler.

2. The apparatus according to claim 1 wherein the housing has a shape ergonomically configured for grasping in a hand of an operator with fingers of the hand wrapped around the housing and a thumb of the hand placed proximal the user interface structure, where the thumb is used to depress and actuate the user interface structure to move the tube coupler in the first direction relative to the housing.

3. The apparatus according to claim 1 wherein movement of the tube coupler in the first direction relative to the housing moves a distal end of the endotracheal tube beyond a distal end of the stylet.

4. The apparatus according to claim 1 further comprising:
a stylet coupler supported by the housing and adapted for slidable motion relative to the housing, the stylet coupler being coupled to the stylet; and
a motive mechanism supported by the housing and adapted to substantially simultaneously move the stylet coupler in a second direction relative to the housing in response to movement of the tube coupler in the first direction relative to the housing.

5. The apparatus according to claim 4 wherein the motive mechanism comprises a pinion gear pivotably coupled to the housing, a tube rack on the tube coupler and having teeth engaging the pinion gear, and a stylet rack on the stylet coupler and having teeth engaging the pinion gear, where the movement of the tube coupler in the first direction drives rotation of the pinion gear by the tube rack, and the rotation of the pinion gear drives translation of the stylet rack causing movement of the stylet coupler in the second direction.

6. The apparatus according to claim 4 wherein the second direction is substantially opposite the first direction.

7. The apparatus according to claim 1 further comprising the endotracheal tube and the stylet, where the proximal end of the endotracheal tube includes a tube connector adapted for connection to the fitting on the second end of the tube coupler prior to intubating the patient.

8. The apparatus according to claim 7 wherein the tube connector includes a first end with a cylinder adapted to fit over a cylinder on the fitting of the tube coupler, and the tube connector includes a second end with a cylinder adapted to fit inside the endotracheal tube.

9. The apparatus according to claim 8 wherein the tube connector further includes flanges on lateral sides, where, when the tube connector is attached to the tube coupler, the flanges snap-fit over bumps on lateral edges of a partial cylindrical wall on the fitting of the tube coupler, and contact between the flanges and the partial cylindrical wall prevents rotation of the tube connector relative to the tube coupler.

10. The apparatus according to claim 9 wherein, after intubating the patient, the tube connector is disconnected from the fitting on the tube coupler, the stylet is fully withdrawn from the endotracheal tube, and the endotracheal tube remains in the airway of the patient.

11. The apparatus according to claim 7 wherein the endotracheal tube includes a groove extending longitudinally along an interior wall at a distal end of the endotracheal tube, and the stylet includes a ridge extending longitudinally along a side at a distal end of the stylet, the ridge on the stylet being shaped to fit in the groove in the interior wall of the endotracheal tube.

12. The apparatus according to claim 11 wherein the endotracheal tube is made with a pre-formed tube curvature along its length and the stylet is made with a pre-formed stylet curvature along its length, and the groove in the interior wall of the endotracheal tube aligns with the ridge on the side of the stylet when the endotracheal tube is loaded onto the stylet with the pre-formed tube curvature oriented opposite the pre-formed stylet curvature.

13. The apparatus according to claim 12 wherein the stylet has a bending stiffness at least ten times greater than a bending stiffness of the endotracheal tube such that the endotracheal tube conforms to the pre-formed stylet curvature when the endotracheal tube is loaded onto the stylet, and engagement of the ridge in the groove prevent rotation of the endotracheal tube relative to the stylet.

14. An apparatus for intubating an airway of a patient, said apparatus comprising:
an endotracheal tube having a proximal end and a distal end, the endotracheal tube including a groove extending longitudinally along an interior wall at the distal end;
a stylet including a ridge extending longitudinally along a side at a distal end of the stylet, the ridge on the stylet being shaped to fit in the groove in the interior wall of the endotracheal tube;
a housing;
a tube coupler supported by the housing and adapted for slidable motion relative to the housing, the tube coupler being connectable to the proximal end of the endotracheal tube,
where the tube coupler is adapted to allow slidable passage of the stylet through an aperture of the tube coupler and into the proximal end of the endotracheal tube,
and where the tube coupler has a first end with a user interface structure operable to move the tube coupler in a first direction relative to the housing in response to actuation of the user interface structure,
and the tube coupler has a second end with a fitting for connection to the proximal end of the endotracheal tube, the fitting being adapted to prevent rotation of the proximal end of the endotracheal tube relative to the tube coupler.

15. The apparatus according to claim 14 wherein the proximal end of the endotracheal tube includes a tube connector adapted for connection to the fitting on the second end of the tube coupler prior to intubating the patient, where the tube connector includes flanges on lateral sides which contact corresponding features of the fitting to prevent rotation of the tube connector relative to the tube coupler.

16. The apparatus according to claim 14 wherein the endotracheal tube is made with a pre-formed tube curvature along its length and the stylet is made with a pre-formed stylet curvature along its length, and the groove in the interior wall of the endotracheal tube aligns with the ridge on the side of the stylet when the endotracheal tube is loaded onto the stylet with the pre-formed tube curvature oriented opposite the pre-formed stylet curvature.

17. The apparatus according to claim 16 wherein the stylet has a bending stiffness at least ten times greater than a bending stiffness of the endotracheal tube such that the endotracheal tube conforms to the pre-formed stylet curvature when the endotracheal tube is loaded onto the stylet, and engagement of the ridge in the groove prevent rotation of the endotracheal tube relative to the stylet.

18. The apparatus according to claim 16 wherein movement of the tube coupler in the first direction relative to the housing moves the distal end of the endotracheal tube beyond the distal end of the stylet.

19. The apparatus according to claim 14 further comprising:
a stylet coupler supported by the housing and adapted for slidable motion relative to the housing, the stylet coupler being coupled to the stylet; and
a motive mechanism supported by the housing and adapted to substantially simultaneously move the stylet coupler in a second direction relative to the housing in response to movement of the tube coupler in the first direction relative to the housing.

20. The apparatus according to claim 19 wherein the motive mechanism comprises a pinion gear pivotably coupled to the housing, a tube rack on the tube coupler and having teeth engaging the pinion gear, and a stylet rack on the stylet coupler and having teeth engaging the pinion gear, where the movement of the tube coupler in the first direction drives rotation of the pinion gear by the tube rack, and the rotation of the pinion gear drives translation of the stylet rack causing movement of the stylet coupler in the second direction.

21. An apparatus for intubating an airway of a patient, said apparatus comprising:
an endotracheal tube having a proximal end and a distal end, the endotracheal tube including a groove extending longitudinally along an interior wall at the distal end;
a stylet including a ridge extending longitudinally along a side at a distal end of the stylet, the ridge on the stylet being shaped to fit in the groove in the interior wall of the endotracheal tube;
a housing;
a tube coupler supported by the housing and adapted for slidable motion in a first direction relative to the housing, the tube coupler being connectable to the proximal end of the endotracheal tube;
a stylet coupler supported by the housing and adapted for slidable motion in a second direction relative to the housing, the stylet coupler being coupled to the stylet; and
a motive mechanism supported by the housing and adapted to substantially simultaneously move the stylet coupler in the second direction relative to the housing in response to movement of the tube coupler in the first direction relative to the housing,
where the tube coupler is adapted to allow slidable passage of the stylet through an aperture of the tube coupler and into the proximal end of the endotracheal tube,
and where the tube coupler has a first end with a user interface structure operable to move the tube coupler in the first direction relative to the housing in response to actuation of the user interface structure,
and the tube coupler has a second end with a fitting for connection to a tube connector on the proximal end of the endotracheal tube, where the tube connector includes flanges on lateral sides which contact corresponding features of the fitting to prevent rotation of the proximal end of the endotracheal tube relative to the tube coupler.

22. The apparatus according to claim 21 wherein the motive mechanism comprises a pinion gear pivotably coupled to the housing, a tube rack on the tube coupler and having teeth engaging the pinion gear, and a stylet rack on the stylet coupler and having teeth engaging the pinion gear, where the movement of the tube coupler in the first direction drives rotation of the pinion gear by the tube rack, and the rotation of the pinion gear drives translation of the stylet rack causing movement of the stylet coupler in the second direction.

23. The apparatus according to claim 21 wherein the endotracheal tube is made with a pre-formed tube curvature along its length and the stylet is made with a pre-formed stylet curvature along its length, and the groove in the interior wall of the endotracheal tube aligns with the ridge on the side of the stylet when the endotracheal tube is loaded onto the stylet with the pre-formed tube curvature oriented opposite the pre-formed stylet curvature.

24. The apparatus according to claim 23 wherein the stylet has a bending stiffness at least ten times greater than a bending stiffness of the endotracheal tube such that the endotracheal tube conforms to the pre-formed stylet curvature when the endotracheal tube is loaded onto the stylet, and engagement of the ridge in the groove prevent rotation of the endotracheal tube relative to the stylet.
